(19) United States
(12) Patent Application Publication
(10) Pub. No.: US 2022/0144749 A1
(43) Pub. Date: May 12, 2022

(54) DOUBLE ALKOXYCARBONYLATION OF DIENES

(71) Applicant: EVONIK OPERATIONS GMBH, Essen (DE)

(72) Inventors: Ji Yang, Harbin City (CN); Matthias Beller, Ostseebad Nienhagen (DE); Ralf Jackstell, Rostock (DE); Robert Franke, Marl (DE)

(73) Assignee: EVONIK OPERATIONS GMBH, Essen (DE)

(21) Appl. No.: 17/523,177

(22) Filed: Nov. 10, 2021

(30) Foreign Application Priority Data
Nov. 12, 2020 (EP) 20207177.5

(51) Int. Cl. C07C 67/38

(52) U.S. Cl. CPC C07C 67/38

(57) ABSTRACT

Process for the double alkoxycarbonylation of dienes.

The invention relates to a process for the double alkoxycarbonylation of dienes.

The alkoxycarbonylation of ethylenically unsaturated compounds is a process of increasing significance. An alkoxycarbonylation is understood to mean the reaction of ethylenically unsaturated compounds (olefins) with carbon monoxide and alcohols in the presence of a metal-ligand complex to give the corresponding esters. Typically, the metal used is palladium. The following scheme shows the general reaction equation for an alkoxycarbonylation:

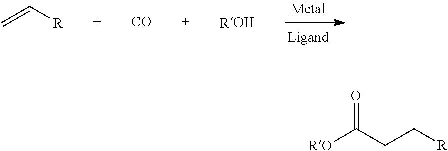

EP 3 121 184 A2 describes a process for the alkoxycarbonylation of olefins using benzene-based diphosphine compounds. In the experiments carried out, methanol (MeOH) is used as solvent.

The technical object of the invention is to provide a novel process that, compared to the prior art mentioned above, provides an increased yield in the double alkoxycarbonylation of dienes.

The object is achieved by a process according to Claim 1.

Process comprising the process steps of:
a) initially charging a diene;
b) adding a ligand of formula (I):

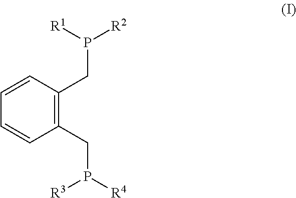

where
R¹, R², R³, R⁴ are selected from: (C₅-C₂₀)-heteroaryl radical, (C₁-C₁₂)-alkyl;
c) adding a compound containing Pd:
d) adding an alcohol,
   wherein the alcohol is added in an amount at least twice that of the diene, based on the molar ratio;
e) adding an organic solvent that is not an alcohol,
wherein the proportion by volume of the solvent, based on the sum of the volumes of the alcohol and solvent, is in the range from 50% by volume to 99.9% by volume;
f) feeding in CO;
g) heating the reaction mixture from steps a) to f), with conversion of the diene into a diester.

It is possible here to add the substances in any order. Typically, however, CO is added after the co-reactants have been initially charged in steps a) to e). In addition, CO can also be fed in in two or more steps, in such a way that, for example, a portion of the CO is first fed in, then the mixture is heated, and then a further portion of CO is fed in.

The expression (C₁-C₁₂)-alkyl encompasses straight-chain and branched alkyl groups having 1 to 12 carbon atoms. These are preferably (C₁-C₈)-alkyl groups, more preferably (C₁-C₆)-alkyl, most preferably (C₁-C₄)-alkyl.

The expression (C₅-C₂₀)-heteroaryl encompasses mono- or polycyclic aromatic hydrocarbon radicals having 5 to 20 carbon atoms, where one or more of the carbon atoms are replaced by heteroatoms. Preferred heteroatoms are N, O and S. The (C₅-C₂₀)-heteroaryl groups have 5 to 20, preferably 5 or 6, ring atoms. Thus, for example, pyridyl is in the context of this invention a C₆-heteroaryl radical and furyl is a C₅-heteroaryl radical.

In one variant of the process, at least two of radicals R¹, R², R³, R⁴ are (C₁-C₁₂)-alkyl.

In one variant of the process, at least two of radicals R¹, R², R³, R⁴ are ^{t}Bu.

In one variant of the process, at least three of radicals R¹, R², R³, R⁴ are (C₁-C₁₂)-alkyl.

In one variant of the process, at least three of radicals R¹, R², R³, R⁴ are ^{t}Bu.

In one variant of the process, the (C₃-C₂₀-heteroaryl radical is 2-pyridyl.

In one variant of the process, the ligand in process step b) has the formula (1);

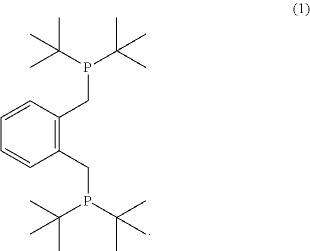 [SMILES: CC(C)(C)P(Cc1ccccc1CP(C(C)(C)C)C(C)(C)C)C(C)(C)C]

In one variant of the process, the Pd-containing compound in process step c) is selected from: palladium(II) trifluoroacetate, palladium dichloride, palladium(II) acetylacetonate, palladium(II) acetate, dichloro(1,5-cyclooctadiene)palladium(II), bis(dibenzylideneacetone)palladium, bis(acetonitrile)dichloropalladium(II), palladium(cinnamyl)dichloride, palladium Iodide, palladium diiodide.

Preferably, the Pd-containing compound is Pd(TFA)₂, Pd(dba)₂, Pd(acac)₂ or Pd(OAc)₂. Particularly suitable are Pd(TFA)₂ and Pd(acac)₂.

The molar ratio of Pd to ligand is preferably in the range from 1:1 to 1:10, preferably from 1:1 to 1:6, more preferably from 1:1 to 1:4.

In one variant of the process, the alcohol in process step d) is selected from: methanol, ethanol, 1-propanol, 1-butanol, 1-pentanol, 1-hexanol, 2-propanol, tert-butanol, 3-pentanol.

In one variant of the process, the alcohol in process step d) is methanol.

In one variant of the process, the solvent in process step e) is selected from: toluene, xylene, anisole, chlorobenzene. THF, methylfuran, propylene carbonate, cyclohexane, alkane, ester, ether.

In one variant of the process, the solvent in process step e) is toluene.

In one variant of the process, the proportion by volume of the solvent, based on the sum of the volumes of the alcohol and solvent, is in the range from 60% by volume to 99.9% by volume.

In one variant of the process, the proportion by volume of the solvent, based on the sum of the volumes of the alcohol and solvent, is in the range from 60% by volume to 99% by volume.

In one variant of the process, the proportion by volume of the solvent, based on the sum of the volumes of the alcohol and solvent, is in the range from 70% by volume to 99% by volume.

In one variant of the process, the proportion by volume of the solvent, based on the sum of the volumes of the alcohol and solvent, is in the range from 70% by volume to 95% by volume.

In one variant of the process, the diene in process step a) is selected from: 1,3-butadiene, 1,2-butadiene, vinylcyclohexene.

In one variant of the process, the process comprises the additional process step h) of:
h) adding a Brønsted acid.

In one variant of the process, the Brønsted acid in process step h) is para-toluenesulfonic acid

CO is in process step f) fed in preferably at a CO partial pressure in the range from 0.1 to 10 MPa (1 to 100 bar), preferably from 1 to 5 MPa (10 to 50 bar), more preferably from 3 to 5 MPa (30 to 50 bar).

In one variant of the process, the reaction mixture is heated in process step g) of the process according to the invention to a temperature in the range from 30° C. to 150° C., preferably from 40° C. to 140° C., more preferably from 60° C. to 130° C., in order to convert the ethylenically unsaturated compound into a diester.

The invention is to be illustrated in detail hereinafter by a working example.

### Experimental Procedures

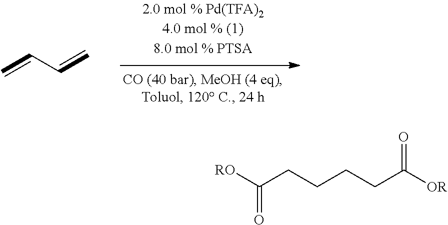

In general, an excess of ligand was used in all catalyst experiments in order to ensure the stability of the active complex at low metal concentration. All experiments used 1,3-butadiene as gas. The butadiene was condensed at low temperature into a special metal tube (this metal tube may be connected to the corresponding autoclave) and the corresponding mass of butadiene accurately weighed to determine the amount added to the autoclave. A 100 ml autoclave was used. First, the autoclave was evacuated and then filled with argon (3× repetition of this process). The solids were then added to the autoclave under an argon atmosphere. Four equivalents of methanol (based on the amount of butadiene) and toluene were used. The volume ratio of methanol and toluene was varied in the series of experiments. The reactions were stirred for 24 hours at the specified temperature. The autoclave was then cooled to room temperature and the pressure cautiously released. Mesitylene (0.5 mmol) was added to the reaction as internal standard. A sample of the mixture was analysed by gas chromatography. Pure product was obtained by column chromatography on silica gel (eluent pentane/ethyl acetate=40).

Reaction Conditions

Alcohol: MeOH (4 equiv. based on the olefin)

Solvent: Toluene

Pressure (CO): 40 bar

Temperature: 120° C.

Reaction time: 24 h

Ligand: (1)

Pd:ligand=2 mol %:4 mol % (based on the olefin)

Experimental Results

| Proportion by | Proportion by | Yield of |
|---|---|---|
| volume of | volume of | diester |
| alcohol [vol.-%] | solvent [vol.-%] | [%] |
| 100 | 0 | 0 |
| 90 | 10 | 0 |
| 80 | 20 | 19 |
| 70 | 30 | 37 |
| 60 | 40 | 42 |
| 50 | 50 | 46 |
| 40 | 60 | 56 |
| 30 | 70 | 71 |
| 20 | 80 | 81 |
| 10 | 90 | 81 |

The proportion by volume of the alcohol and of the solvent is based on the sum of the volumes of the alcohol and solvent (=100% by volume).

1. Process comprising the process steps of:
a) initially charging a diene;
b) adding a ligand of formula (I): 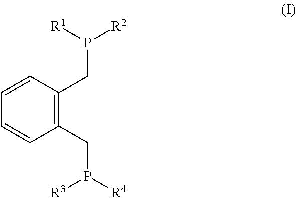
where
R¹, R², R³, R⁴ are selected from: (C₅-C₂₀)-heteroaryl radical, (C₁-C₁₂)-alkyl;
c) adding a compound containing Pd;
d) adding an alcohol,
wherein the alcohol is added in an amount at least twice that of the diene, based on the molar ratio;
e) adding an organic solvent that is not an alcohol,
wherein the proportion by volume of the solvent, based on the sum of the volumes of the alcohol and solvent, is in the range from 50% by volume to 99.9% by volume;
f) feeding in CO;
g) heating the reaction mixture from steps a) to f), with conversion of the diene into a diester.

2. Process according to claim 1,
wherein at least two of radicals R¹, R², R³, R⁴ are (C₁-C₁₂)-alkyl.

3. Process according to claim 1,
wherein at least two of radicals R¹, R², R³, R⁴ are ^{t}Bu.

4. Process according to claim 1,
wherein at least three of radicals R¹, R², R³, R⁴ are (C₁-C₁₂)-alkyl.

5. Process according to claim 1,
wherein at least three of radicals R¹, R², R³, R⁴ are ^{t}Bu.

6. Process according to claim 1,
wherein the (C₃-C₂₀)-heteroaryl radical is 2-pyridyl.

7. Process according to claim 1,
wherein the ligand in process step b) has the formula (1):

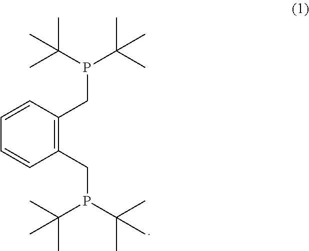 [SMILES: CC(C)(C)P(Cc1ccccc1CP(C(C)(C)C)C(C)(C)C)C(C)(C)C]

8. Process according to claim 1,
wherein the Pd-containing compound in process step c) is selected from: palladium(II) trifluoroacetate, palladium dichloride, palladium(II) acetylacetonate, palladium(II) acetate, dichloro(1,5-cyclooctadiene)palladium(II), bis(dibenzylideneacetone)palladium, bis(acetonitrile)dichloropalladium(II), palladium(cinnamyl)dichloride, palladium iodide, palladium diiodide.

9. Process according to claim 1,
wherein the alcohol in process step d) is selected from: methanol, ethanol, 1-propanol, 1-butanol, 1-pentanol, 1-hexanol, 2-propanol, tert-butanol, 3-pentanol.

10. Process according to claim 1,
wherein the alcohol in process step d) is methanol.

11. Process according to claim 1,
wherein the solvent in process step e) is selected from: toluene, xylene, anisole, chlorobenzene, THF, methylfuran, propylene carbonate, cyclohexane, alkane, ester, ether.

12. Process according to claim 1,
wherein the solvent in process step e) is toluene.

13. Process according to claim 1,
wherein the proportion by volume of the solvent, based on the sum of the volumes of the alcohol and solvent, is in the range from 60% by volume to 99.9% by volume.

14. Process according to claim 1,
wherein the diene in process step a) is selected from: 1,3-butadiene, 1,2-butadiene, vinylcyclohexene.

15. Process according to claim 1,
wherein the process comprises the additional process step h) of:
h) adding a Brønsted acid.
